(19) United States
(12) Patent Application Publication
(10) Pub. No.: US 2011/0213191 A1
(43) Pub. Date: Sep. 1, 2011

(54) COMPOSITIONS AND METHODS FOR OLEFIN RECOVERY

(75) Inventors: Robert C. Schucker, The Woodlands, TX (US); Michael F. Lynch, Houston, TX (US)

(73) Assignee: Trans Ionics Corporation, The Woodlands, TX (US)

(21) Appl. No.: 12/997,148

(22) PCT Filed: Jun. 8, 2009
(86) PCT No.: PCT/US09/46559

§ 371 Date: May 12, 2011

Related U.S. Application Data
(60) Provisional application No. 61/060,056, filed on Jun. 9, 2008.
(60) Provisional application No. 61/060,045, filed on Jun. 9, 2008.
(60) Provisional application No. 61/060,052, filed on Jun. 9, 2008.
(60) Provisional application No. 61/060,044, filed on Jun. 9, 2008.

(51) Int. Cl. C07C 7/156; C07C 7/152; C09K 3/00

(52) U.S. Cl. 585/845; 585/855; 252/184

(57) ABSTRACT

The present invention is directed to compositions and methods for the recovery of olefins from a mixture. The compositions of the present invention comprise: (1) a transition metal ion; (2) a counter anion; (3) a ligand selected from the group consisting of a bidentate ligand and a tridentate ligand, wherein the ligand comprises at least two nitrogen atoms, and wherein each of the nitrogen atoms comprises a lone pair of electrons; and (4) a polar solvent with a boiling point of at least about 200° C. The methods of the present invention comprise: (1) providing the aforementioned compositions; (2) bonding at least a portion of the olefins in a mixture to the transition metal ion in the composition to form a complex; (3) separating the complex from the mixture; and (4) recovering the olefins from the complex.

### STATEMENT REGARDING FEDERALLY SPONSORED RESEARCH

The United States Government has rights in this invention pursuant to Contract No. DE-FG02-05ER84262 between the United States Department of Energy and Trans Ionics Corporation.

### FIELD OF THE INVENTION

The present invention relates to compositions capable of selectively and reversibly binding olefins, thereby facilitating their separation from mixtures, such as olefin/paraffin mixtures in gaseous and/or liquid streams.

### BACKGROUND

Many olefins, such as ethylene and propylene, can be produced by various processes operated by the chemical and refining industries. One of such processes is steam cracking of feeds such as ethane, propane, butane, naphtha or gas oil. A preferred feed stock for such a process is the natural gas liquids (NGL) stream because of high yields of desired products. Another process involves the recovery of light ends from fluid catalytic cracking. In both such cases, however, the products of the conversion reactors are mixtures of chemical species that require additional separation and purification steps.

Traditionally, additional separation and purification steps of olefins have been done by distillation. For instance, the separation of ethylene from ethane or propylene from propane by distillation has been typically accomplished under cryogenic conditions at elevated pressures due to the low boiling points of these liquids. Cryogenic distillation, however, is extremely energy intensive, resulting in substantial costs to separate olefins from paraffins. For instance, it has been estimated that such separations may account for 6.3% (about 0.15 quadrillion BTUs) of the energy used by the chemical and petrochemical industries.

Furthermore, there are numerous examples of mixed liquid olefin/paraffin streams that cannot be effectively separated by distillation because of similarities in boiling points. One example of such a stream is a byproduct of the synthesis of ethylene-1-octene copolymer, which comprises a mixture of a paraffinic solvent and more than a dozen C₈ olefins, which cannot be separated by distillation.

Therefore, there is currently a need for alternative olefin separation methods that are less energy intensive than those presently used in the art. There is also a need for more effective methods to separate olefins from other compounds in a mixture, particularly compounds with similar boiling points.

### SUMMARY

In some embodiments, the present invention provides a composition for the recovery of olefins from a mixture. Such compositions comprise: (1) a transition metal ion; (2) a counter anion; (3) a ligand selected from the group consisting of a bidentate ligand and a tridentate ligand, where the ligand comprises at least two nitrogen atoms, and where each of the nitrogen atoms comprises a lone pair of electrons; and (4) a polar solvent with a boiling point of at least about 200° C.

In other embodiments, the present invention provides methods for recovering olefins from a mixture, where the methods comprise: (1) providing the aforementioned composition; (2) bonding at least a portion of the olefins in the mixture to the transition metal ion in the composition to form a complex; (3) separating the complex from the mixture; and (4) recovering the olefins from the complex.

In various embodiments, the transition metal ions of the compositions may be Cu⁺. Likewise, the counter anions of the compositions may be selected from the group consisting of PF₆⁻¹, BF₄⁻¹, NO₃⁻¹, BPh₄⁻¹, Cl⁻¹, I⁻¹, Br⁻¹, F⁻¹, and COO⁻.

In further embodiments, the ligands may have at least two aromatic rings, where each of the aromatic rings comprise a nitrogen atom with a lone pair of electrons. In other embodiments, the ligand may be a bidentate ligand selected from the group consisting of 2,2′-dipyridyl amine, 2,2′-dipyridyl ketone and 2,2′-dipyridyl methane. In further embodiments, the ligand may be a tridentate ligand selected from the group consisting of terpyridine and di-(2-picolylamine).

In other embodiments of the present invention, the solvent may comprise a polyalkylene glycol selected from the group consisting of diethylene glycol, triethylene glycol, tetraethylene glycol, pentaethylene glycol and hexaethylene glycol. In further embodiments, the solvent may comprise an ionic liquid selected from the group consisting of 1-butyl-3-methylimidazolium hexafluorophosphate, 1-ethyl-3-methylimidazolium tetrachloroaluminate, 1-butylpyridinium nitrate, 1-butyl-3-methylimidazolium tetrafluoroborate and mixtures thereof.

### BRIEF DESCRIPTION OF THE DRAWINGS

For a more complete understanding of the present disclosure, and the advantages thereof, reference is now made to the following descriptions to be taken in conjunction with the accompanying drawings describing specific embodiments of the disclosure, wherein:

FIG. 1 provides the structures of several bidentate and tridentate ligands as non-limiting examples of ligands that can be used with the compositions of the present invention.

FIG. 2 provides depictions of the dₓ²_{-y}² and d_{z}² orbitals of various transition metals, such as Cu⁺. Without being bound by theory, it is envisioned that the dₓ²_{-y}² and d_{z}² orbitals of the transition metal ions of the present invention are involved in complex formation with olefins.

### DETAILED DESCRIPTION

In the following description, certain details are set forth such as specific quantities, concentrations, sizes, etc. so as to provide a thorough understanding of the various embodiments disclosed herein. However, it will be apparent to those skilled in the art that the present disclosure may be practiced without such specific details. In many cases, details concerning such considerations and the like have been omitted inasmuch as such details are not necessary to obtain a complete understanding of the present disclosure and are within the skills of persons of ordinary skill in the relevant art.

The present invention is directed at compositions and methods for the recovery of olefins from a mixture. Such mixtures may be olefin/paraffin mixtures. Such mixtures may also be feed streams, such gaseous and/or liquid streams. For instance, in some embodiments, the mixture is in a gaseous phase. In other embodiments, the mixture is in a liquid phase. In further embodiments, the olefin to be recovered in the mixture comprises an unsaturated hydrocarbon.

The compositions of the present invention generally comprise: (1) a transition metal ion; (2) a counter anion; (3) a ligand selected from the group consisting of a bidentate ligand and a tridentate ligand, wherein the ligand comprises at least two nitrogen atoms, and wherein each of the nitrogen atoms comprises a lone pair of electrons; and (4) a polar solvent with a boiling point of at least about 200° C.

Transition Metal Ions

In some embodiments, the transition metal ion of the compositions of the present invention is Cu⁺. Such a Cu⁺ ion in the present invention may be obtained in a number of non-limiting ways. For instance, the Cu⁺ ion may be obtained from cuprous salts, such as CuCl, CuI, CuBr or CuCN. However, though such salts are readily available, they may not always be soluble in a solvent of choice for various embodiments of the present invention. Therefore, in other embodiments, Cu⁺ coordination complexes with acetonitrile may be purchased commercially for use as a transition metal ion. Such complexes usually consist of Cu⁺ ions coordinated in all four available positions with acetonitrile and a fixed anion such as the hexafluorophosphate ion (PF₆⁻¹). This material is referred to as tetrakis(acetonitrile)copper(I) hexafluorophosphate. In solution, the monodentate acetonitrile ligands are easily exchanged for more stable bidentate or tridentate ligands.

In other embodiments, Cu⁺ may be made in-situ by reducing a Cu⁺⁺ salt such as Cu(NO₃)₂.2.5 H₂O with elemental copper (Cu⁰) in acetonitrile to form tetrakis(acetonitrile)copper(I) nitrate. However, it should be recognized that anyone skilled in the art may select other salts that may produce an acceptable Cu(I) coordination complex with any number of ligands.

In other embodiments, the transition metal ion of the compositions of the present invention is Ag⁺. Such a Ag⁺ ion in the present invention may also be obtained in a number of non-limiting ways, as known by persons of ordinary skill in the art.

Furthermore, Applicants note that the aforementioned transition metal ions are only specific and non-limiting examples of transition metal ions that may be used in the present invention. Thus, a person of ordinary skill in the art can envision additional suitable transition metal ions that fall within the scope of the present invention that were not disclosed here.

Counter Anions

In some embodiments, counter anions that are suitable for use in the compositions of the present invention include but are not limited to hexafluorophosphate (PF₆⁻¹), tetrafluoroborate (BF₄⁻¹), nitrate (NO₃⁻¹) and tetraphenylborate (BPh₄⁻¹). By way of example, and without being bound by theory, the selection of counter anions in the present invention may be based on measurable interactions. For example, tetrafluoroborate has the possibility of a B—P . . . Cu interaction that may compete with the Cu . . . olefin binding. However, the equivalent interaction for tetraphenylborate (i.e., Ph . . . Cu) may be weaker.

In further embodiments, counter anions suitable for use in the compositions of the present invention may also be simple halides, such as chloride (Cl⁻¹), iodide (I⁻¹), bromide (Br⁻¹) and fluoride (F⁻¹). In further embodiments, counter anions may be carboxylate anions (COO⁻). However, the aforementioned halides and carboxylate anions may also be capable of competing as ligands due to their lone pair of electrons. Accordingly, compositions made using such species may, at least in some embodiments, undergo disproportionation to Cu⁺⁺ and Cu⁰.

In other embodiments, the counter anion is selected from the group consisting of PF₆⁻¹, BF₄⁻¹, NO₃⁻¹, BPh₄⁻¹, Cl⁻¹, I⁻¹, Br⁻¹, F⁻¹, and COO⁻. In various embodiments, the counter anion comprises a non-coordinating anion. Applicants also note that the aforementioned counter anions are only specific and non-limiting examples of counter anions that may be used in the present invention. Thus, a person of ordinary skill in the art can envision additional suitable counter anions that fall within the scope of the present invention that were not disclosed here.

Ligands

By way of background, transition metal ions are Lewis acids that form stable Lewis Acid-Base adducts with Lewis bases. Ligands are Lewis bases because they bear at least one atom having a lone pair of electrons. For instance, ligands such as H₂O, NH₃, CO, OH⁻¹, and CN⁻¹ that bear a single Lewis base atom are termed monodentate ligands. Likewise, ligands bearing two such atoms are termed bidentate ligands. Similarly, ligands that bear three Lewis base atoms are termed tridentate ligands.

Monodentate ligands such as pyridine can interact with Cu⁺ to form a copper complex that can be used in the compositions to separate olefins. Such monodentate copper complexes are often unstable, however. Tetradentate ligands, in which the lone pairs are separated by several intervening atoms, can occupy all four dₓ²_{-y}² orbitals of a transition metal ion to form stable complexes known as chelates. Such chelate complexes may not have the ability to interact with electrons from an olefin for binding and separation to occur. Likewise, polydentate ligands that contain more than four lone pairs of electrons have the same olefin binding limitations. However, such limitations generally do not apply to bidentate or tridentate ligands.

Accordingly, in an embodiment, ligands suitable for use with the compositions of the present invention are selected from the group consisting of bidentate and tridentate ligands. Such bidentate and tridentate ligands desirably comprise at least two nitrogen atoms, each with a lone pair of electrons. In other embodiments, the bidentate or tridentate ligand may comprise two or more aromatic rings, where each of the aromatic rings may comprise at least one nitrogen atom with a lone pair of electrons. In other embodiments, the aromatic rings may be connected to each other by carbon or nitrogen linkages. For instance, a general structure for a ligand suitable for use with the compositions of the present invention is shown below as a non-limiting example:

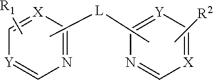

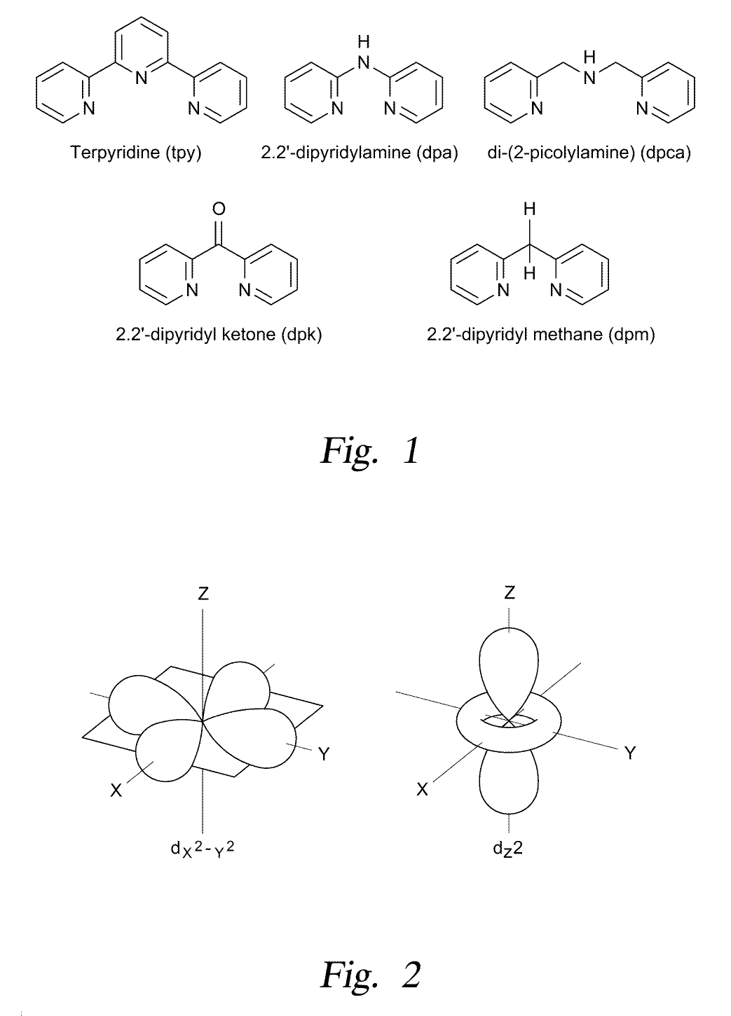

In this generalization, X and Y represent either carbon (C) or nitrogen (N). Likewise, R₁ and R₂ represent substituents on the aromatic rings at any allowable position. Such substituents may be alkyl or aromatic in nature. In addition, L represents a linking group which may comprise any of the groups shown below:

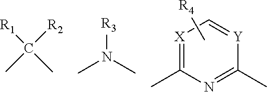

where R₁, R₂, and R₄ represent substituents that may comprise: (1) a single atom such as H, F, Cl, Br or I; (2) an alkyl group; or (3) an aromatic ring. Likewise, R₃ represents substituents that may comprise: (1) a single atom such as H; (2) an alkyl group; or (3) an aromatic ring. Non-limiting examples of such ligands are shown in FIG. 1.

A person of ordinary skill in the art will recognize that numerous ligands may be suitable for use in the present invention. Furthermore, such ligand may have various physical properties. For instance, in some embodiments, the ligand is a bidentate ligand. In additional embodiments, the bidentate ligand has a boiling point of at least about 200° C. In further embodiments, the bidentate ligand has a vapor pressure of less than about 0.01 kPa at 20° C. However, in additional embodiments, the bidentate ligand may have a vapor pressure of less than about 0.005 kPa at 20° C., or less than about 0.001 kPa at 20° C. In more specific embodiments, the bidentate ligand comprises at least two aromatic rings, wherein each of the aromatic rings comprises a nitrogen atom with a lone pair of electrons. In additional embodiments, the bidentate ligand is selected from the group consisting of 2,2′-dipyridyl amine, 2,2′-dipyridyl ketone and 2,2′-dipyridyl methane.

In other embodiments, the ligand is a tridentate ligand. In additional embodiments, the tridentate ligand has a boiling point of at least about 200° C. In further embodiments, the tridentate ligand has a vapor pressure of less than about 0.01 kPa at 20° C. However, in other embodiments, the tridentate ligand may have a vapor pressure of less than about 0.005 kPa at 20° C., or less than about 0.001 kPa at 20° C. In more specific embodiments, the tridentate ligand comprises at least two aromatic rings, wherein each of the aromatic rings comprises a nitrogen atom with a lone pair of electrons. In additional embodiments, the tridentate ligand is selected from the group consisting of terpyridine and di-(2-picolylamine).

The chemical structures of exemplary bidentate and tridentate ligands are shown in FIG. 1 as non-limiting examples. However, Applicants note that the ligands shown in FIG. 1 and described in this specification are only specific and non-limiting examples of ligands that may be used in the present invention. Thus, a person of ordinary skill in the art can envision additional suitable ligands that fall within the scope of the present invention that were not disclosed here.

Solvents

Various solvents may be used with the compositions of the present invention. In some embodiments, the solvent is a high boiling solvent (i.e., a solvent with a high boiling point, such as a boiling point of at least about 200° C.). In other embodiments, the solvent is a polar solvent with acceptable electronic properties (e.g., dipole moment, polarizability, etc.).

In further embodiments, the solvent may also have low a vapor pressure. For instance, in some embodiments, the solvent has a vapor pressure of less than about 0.01 kPa at 20° C. In other embodiments, the solvent may have a vapor pressure of less than about 0.1 kPa at 20° C., less than about 0.05 kPa at 20° C., or less than about 0.005 kPa at 20° C.

In other embodiments, the solvent may have one or more of the following physical properties: (1) a boiling point greater than about 200° C.; (2) a vapor pressure of less than about 0.005 kPa at 20° C.; and (3) a viscosity lower than 100 mPa·s at 25° C.

In other embodiments, the boiling point of the solvent is higher than the boiling point of the highest boiling olefin in the mixture. For instance, in some embodiments, the boiling point of the solvent is at least about 20° C. higher than the boiling point of the highest boiling olefin in the mixture. In other embodiments, the boiling point of the solvent is at least about 50° C. higher than the boiling point of the highest boiling olefin in the mixture. In still other embodiments, the boiling point of the solvent is at least about 100° C. higher than the boiling point of the highest boiling olefin in the mixture.

A non-limiting example of a solvent suitable for use with the compositions of the present invention may be a polyalkylene glycol with the following general formula:

H—(O—CH₂CH₂)ₙ—OH:2≦n≦10.

In various embodiments, n represents a value ranging from 2 to 10. However, in other embodiments, n may have different value ranges. In more specific embodiments, n represents a value ranging from 2 to 6. In other embodiments, the polyalkylene glycol is selected from the group consisting of diethylene glycol, triethylene glycol, tetraethylene glycol, pentaethylene glycol and hexaethylene glycol.

In additional embodiments, solvents may be an adiponitrile. In other embodiments, the solvent comprises an ionic liquid. In more specific embodiments, the ionic liquid is selected from the group consisting of 1-butyl-3-methylimidazolium hexafluorophosphate, 1-ethyl-3-methylimidazolium tetrachloroaluminate, 1-butylpyridinium nitrate, 1-butyl-3-methylimidazolium tetrafluoroborate and mixtures thereof.

Applicants also note that the aforementioned solvents are only specific and non-limiting examples of solvents that may be used in the present invention. Thus, a person of ordinary skill in the art can envision additional suitable solvents that fall within the scope of the present invention that were not disclosed here:

Methods for Recovering Olefins from a Mixture

The present invention also provides methods for recovering olefins from a mixture. In various embodiments, the methods comprise:
(1) providing a composition that comprises: (a) a transition metal ion; (b) a counter anion; (c) a ligand selected from the group consisting of a bidentate ligand and a tridentate ligand, wherein the ligand comprises at least two nitrogen atoms, and wherein each of the nitrogen atoms comprises a lone pair of electrons; and (d) a polar solvent with a boiling point of at least about 200° C.;
(2) bonding at least a portion of the olefins in the mixture to the transition metal ion in the composition to form a complex;
(3) separating the complex from the mixture; and
(4) recovering the olefins from the complex.

Various compositions may be used with the methods of the present invention for recovering olefins from a mixture. For instance, in some embodiments, the transition metal ion in the composition is Cu⁺. In other embodiments, the ligand in the composition is a bidentate ligand with at least two aromatic rings, wherein each of the aromatic rings comprises a nitrogen atom with a lone pair of electrons. Likewise, in other embodiments, the ligand in the composition is a tridentate ligand with at least two aromatic rings, wherein each of the aromatic rings comprises a nitrogen atom with a lone pair of electrons.

Likewise, the above-described bonding of the olefins in the mixture to the transition metal in the composition can occur under various reaction conditions. For instance, in some embodiments, the reaction conditions include mixing the composition with the mixture. In some embodiments, the mixing comprises stirring.

The above-described separation step of the transition metal ion-olefin complex can also occur by various methods. For instance, in some embodiments, the separation step comprises phase separation. In other embodiments, the phase separation comprises incubating the complex and the mixture at room temperature. In other embodiments, the phase separation comprises centrifugation.

Similarly, the above-described recovery step of olefins from the transition metal ion-olefin complex can occur by numerous methods. For instance, in some embodiments, the recovery comprises reducing pressure. Without being bound by theory, it is envisioned that a reduction in pressure volatilizes the olefins away from the relatively nonvolatile solvent complexing agent.

Applicants also note that the aforementioned method for recovering olefins from a mixture are only non-limiting examples. Thus, a person of ordinary skill in the art can envision additional suitable methods that fall within the scope of the present invention that were not disclosed here.

### EXAMPLES

The following experimental examples are included to demonstrate particular aspects of the present disclosure. It should be appreciated by those of skill in the art that the examples that follow merely represent exemplary embodiments of the disclosed compositions and methods for recovering olefins. Therefore, those of skill in the art should, in light of the present disclosure, appreciate that many changes can be made in the specific embodiments described and still obtain a like or similar result without departing from the spirit and scope of the present disclosure.

### Example 1

To a 250 ml round bottom flask, equipped with a stirrer and placed in a constant temperature oil bath, were added 50.0 g of tetraethylene glycol (TEG) and 25.0 g of a mixed olefin/paraffin feed having the composition of olefins shown in Table 1 with the balance of the feed being Isopar E, a paraffinic solvent sold by ExxonMobil Corporation. The mixture was stirred for two hours at 50° C., whereupon the stirring was discontinued and the phases allowed to separate. Approximately 1 ml of each phase was removed using a 2 ml syringe and the samples were analyzed by gas chromatography.

TABLE 1

| | | | Percent |
|---|---|---|---|
| Component | Feed | Raffinate | Decrease |
| 2-ethyl-1-hexene | 0.08% | 0.10% | −19.20% |
| 1-octene | 13.56% | 13.42% | 1.06% |
| cis-3-methyl-3-heptene | 0.24% | 0.28% | −16.26% |
| trans-4-octene | 1.31% | 1.32% | −0.08% |
| trans-3-methyl-2-+trans-3- | 4.17% | 4.13% | 1.13% |
| methyl-3-heptene, trans-3-octene | | | |
| trans-2-octene | 5.59% | 5.58% | 0.11% |
| cis-3-methyl-2-heptene | 1.16% | 1.15% | 1.59% |
| cis-2-octene | 4.42% | 4.39% | 0.64% |
| total olefins: | 30.54% | 30.35% | 0.61% |
| total non-olefins: | 69.46% | 69.65% | |

Results shown in Table 1 indicated that TEG by itself had a low capacity for all of the hydrocarbons and a low selectivity for olefins, reducing the 1-octene concentration in the feed by only 1%.

### Example 2

To a 100 ml round bottom flask was added 0.65 g cupric nitrate and 17.04 g acetonitrile (purged with nitrogen for 30 min), affording a clear blue solution. To this was then added 0.30 g copper powder. This mixture stirred for one hour. To a 50 ml Schlenk flask was added 0.96 g 2,2′-dipyridyl amine (dpy) and 15.76 g TEG. This produced a clear yellow solution after stirring. The clear colorless cuprous nitrate solution was filtered through a flitted filter funnel and into the ligand solution, producing a clear, very light orange solution. This flask was placed under vacuum for one hour until all acetonitrile had been removed. Then to the remaining clear orange solution was added 1.58 g of a mixed olefin/paraffin feed having the composition shown in Table 2. The mixture was stirred vigorously for thirty minutes. Over this time the solution darkened slightly to a light green color. The mixture was allowed to phase separate and a sample of the raffinate was analyzed by gas chromatography. Results are shown in Table 2.

TABLE 2

| | | | Percent |
|---|---|---|---|
| Component | Feed | Raffinate | Decrease |
| 2-ethyl-1-hexene | 0.08% | 0.08% | −2.14% |
| 1-octene | 11.98% | 3.12% | 73.99% |
| cis-3-methyl-3-heptene | 0.26% | 0.51% | −95.00% |
| trans-4-octene | 1.21% | 1.15% | 4.70% |
| trans-3-methyl-2-+trans-3- | 3.83% | 3.64% | 4.74% |
| methyl-3-heptene, trans-3-octene | | | |
| trans-2-octene | 4.87% | 4.46% | 8.34% |
| cis-3-methyl-2-heptene | 1.10% | 1.10% | −0.02% |
| cis-2-octene | 3.94% | 2.98% | 24.38% |
| total olefins: | 27.25% | 17.03% | 37.496 |

As can be seen, a composition having the composition of the present invention comprising (1) Cu⁺, (2) a nitrate (NO₃) anion, (3) 2,2′-dipyridyl amine as a ligand in (4) a high boiling solvent (TEG) removed 74% of the 1-octene and 37% of the total olefins from the feed in a single stage.

### Example 3

To a 100 ml Schlenk flask was added 18.06 g acetonitrile. This was degassed via three freeze pump thaw cycles. Then to the stirring solvent was added 1.03 g cupric nitrate trihydrate and 0.35 g copper powder. This mixture was stirred with heating and refluxed in the flask for two hours. The clear copper solution with a small amount of unreacted copper and some blue colored precipitate was filtered through a fitted filter funnel into a flask containing 15.33 g of TEG. To the resulting clear colorless solution was added 1.74 g di-(2-picolyl amine). This resulted in a clear light brown solution. A vacuum was applied and the solution heated. The acetonitrile then was removed by pulling a vacuum on the approximately 100° C. solution over the course of three hours. As the acetonitrile came off, the solution darkened considerably, to a final dark brown color. The vacuum and heating were stopped before all of the acetonitrile came off. Once the solution returned to room temperature, a sample of 1.54 g mixed olefin/paraffin feed was added. This was stirred vigorously for 30 minutes. The stirring was stopped and allowed to phase separate, whereupon a sample of the raffinate taken for analysis by gas chromatography. The results are shown in Table 3.

TABLE 3

| | | | Percent |
|---|---|---|---|
| Component | Feed | Raffinate | Decrease |
| 2-ethyl-1-hexene | 0.08% | 0.05% | 39.491 |
| 1-octene | 11.98% | 3.08% | 74.316 |
| cis-3-methyl-3-heptene | 0.26% | 0.20% | 24.404 |
| trans-4-octene | 1.21% | 1.04% | 13.924 |
| trans-3-methyl-2-+trans-3- | 3.83% | 3.35% | 12.338 |
| methyl-3-heptene, trans-3-octene | | | |
| trans-2-octene | 4.87% | 4.11% | 15.541 |
| cis-3-methyl-2-heptene | 1.10% | 1.08% | 1.539 |
| cis-2-octene | 3.94% | 2.20% | 44.05 |
| total olefins: | 27.25% | 15.38% | 43.562 |

As can be seen, a composition having the composition of the present invention comprising (1) Cu⁺, (2) a nitrate (NO₃)⁻ anion, (3) di-(2-picolyl amine) as a ligand in (4) a high boiling solvent (TEG) removed 74% of the 1-octene and 44% of the total olefins from the feed in a single stage.

### Example 4

Into a 50 ml Schlenk flask, in the glove box, was added 0.982 g (0.00293 mol) Cu(II)(BF₄)₂. This was dissolved in 13.771 g acetonitrile. This resulted in a blue, slightly cloudy mixture. This was removed from the glove box and into the stirring solution was placed 0.40 g (0.0063 mol) washed copper powder. The mixture was heated to reflux and stirred for 2 hours. Into a separate 50 ml Schlenk flask in the glove box was added 1.518 g (0.00887 mol) 2,2′-dipyridyl amine (dpy). To this was added 15.608 g TEG. The flask was removed from the glove box and a vacuum was pulled on the mixture while stirring. The yellow solid slowly dissolved, yielding a clear bright yellow solution. The vacuum was broken with nitrogen; and a fritted filter funnel was poised above the flask. The cooled clear colorless Cu(I) solution was filtered from the unreacted copper through the frit. Upon completion of this addition, the resulting clear light orange solution was placed under vacuum and heated to remove the acetonitrile. As the acetonitrile was removed over the course of 1.25 hours under vacuum the solution became a slightly darker orange and more viscous. Once all acetonitrile had been removed, to the clear burnt orange colored solution was added 1.68 g of a mixed olefin/paraffin feed having the composition shown in Table 4. This was stirred vigorously for 30 minutes with no apparent color change or solid formation. The two phases were allowed to separate and a sample of the raffinate was analyzed by gas chromatography. The results are shown in Table 4.

TABLE 4

| | | | Percent |
|---|---|---|---|
| Component | Feed | Raffinate | Decrease |
| 2-ethyl-1-hexene | 0.08% | 0.06% | 19.545 |
| 1-octene | 11.98% | 5.38% | 55.101 |
| cis-3-methyl-3-heptene | 0.26% | 0.71% | |
| trans-4-octene | 1.21% | 1.18% | 2.144 |
| trans-3-methyl-2-+trans-3- | 3.83% | 3.76% | 1.707 |
| methyl-3-heptene, trans-3-octene | | | |
| trans-2-octene | 4.87% | 4.66% | 4.248 |
| cis-3-methyl-2-heptene | 1.10% | 1.07% | 2.213 |
| cis-2-octene | 3.94% | 3.45% | 12.467 |
| total olefins: | 27.25% | 20.27% | 25.622 |

As can be seen, a composition having the composition of the present invention comprising (1) Cu⁺, (2) a tetrafluoroborate (BF₄)⁻ anion, (3) 2,2′-dipyridyl amine as a ligand in (4) a high boiling solvent (TEG) removed 55% of the 1-octene and 26% of the total olefins from the feed in a single stage.

### Example 5

To a 50 ml Schlenk flask in the glove box was added 0.235 g CuCl. To this was added 18.402 g TEG. This mixture was allowed to stir 30 minutes. There remained undissolved solid and a green solution. Then to this mixture was added 0.505 g 2,2′-dipyridyl amine. The solid in the flask began to dissolve with the addition of the ligand, and the solution appeared as a clear, light brown/orange color. To this solution was added 1.705 g of a mixed olefin/paraffin feed having the composition shown in Table 5. This mixture was stirred vigorously for 30 minutes. A sample of the raffinate was analyzed by gas chromatography and produced the results shown in Table 5.

TABLE 5

| | | | Percent |
|---|---|---|---|
| Component | Feed | Raffinate | Decrease |
| 2-ethyl-1-hexene | 0.08% | 0.08% | −9.371 |
| 1-octene | 11.98% | 11.25% | 6.063 |
| cis-3-methyl-3-heptene | 0.26% | 0.29% | −11.03 |
| trans-4-octene | 1.21% | 1.17% | 2.798 |
| trans-3-methyl-2-+trans-3- | 3.83% | 3.65% | 4.638 |
| methyl-3-heptene, trans-3-octene | | | |
| trans-2-octene | 4.87% | 4.70% | 3.568 |
| cis-3-methyl-2-heptene | 1.10% | 1.01% | 8.325 |
| cis-2-octene | 3.94% | 3.70% | 5.973 |
| total olefins: | 27.25% | 25.85% | 5.146 |

As can be seen, a composition having a composition of the present invention comprising (1) Cu⁺, (2) a chloride (Cl⁻) anion, (3) 2,2′-dipyridyl amine as a ligand in (4) a high boiling solvent (TEG) still removed 6% of the 1-octene and 5% of the total olefins from the feed in a single stage, considerably more than TEG alone.

### Example 6

To a 100 ml Schlenk flask was added 13.65 g acetonitrile. This was degassed via three freeze/pump/thaw cycles. To the stirring acetonitrile was added 0.90 g copper(II) tetrafluoroborate hydrate and 0.40 g Cu powder. This mixture was brought to reflux and stirred under nitrogen for one hour. In the glove box was placed a separate 50 ml Schlenk flask. To this was added 14.30 g TEG and 1.24 g 2,2′-dipyridyl amine. This mixture was taken from the glove box and stirred under vacuum for 10 minutes as the solid dissolved and the solvent deoxygenated. The clear Cu(I) solution that resulted from the reduction of the cupric tetrafluoroborate was filtered through a fritted filter funnel and into the stirring yellow ligand solution. The resulting clear yellow/orange solution was stirred under vacuum for 2 hours. Once all of the acetonitrile was removed, the resulting clear orange solution was allowed to cool to room temp. As the solution cooled, some white/yellow colored precipitate began to come out of the solution. An additional 1.5 rill of acetonitrile was added and the solid went back into solution. At this point, a sample of 1.65 g of a mixed olefin/paraffin feed having the composition shown in Table 6 was added with vigorous stirring. The stirring was stopped after 30 minutes and the two phases allowed to separate. A sample of the raffinate was analyzed by gas chromatography and produced the results shown in Table 6.

TABLE 6

| | | | Percent |
|---|---|---|---|
| Component | Feed | Raffinate | Decrease |
| 2-ethyl-1-hexene | 0.08% | 0.08% | 18.608 |
| 1-octene | 11.98% | 8.83% | 26.322 |
| cis-3-methyl-3-heptene | 0.26% | 0.28% | −8.955 |
| trans-4-octene | 1.21% | 1.17% | 3.435 |
| trans-3-methyl-2-+trans-3- | 3.83% | 3.69% | 3.501 |
| methyl-3-heptene, trans-3-octene | | | |
| trans-2-octene | 4.87% | 4.63% | 5.05 |
| cis-3-methyl-2-heptene | 1.10% | 1.03% | 6.203 |
| cis-2-octene | 3.94% | 3.59% | 8.852 |
| total olefins: | 27.25% | 23.87% | 14.612 |

As can be seen, a composition having the composition of the present invention comprising (1) Cu⁺, (2) a tetrafluoroborate (BF₄)⁻ anion, (3) 2,2′-dipyridyl amine as a ligand in (4) a high boiling solvent (TEG) removed 26% of the 1-octene and 15% of the total olefins from the feed in a single stage. Without being bound by theory, it is envisioned that the addition of excess acetonitrile to bring the solids back into solution may have decreased the capacity of the complex for the olefins.

The two phase solution remaining in the flask was placed under vacuum and heated for about 10 minutes. When it appeared that all of the raffinate phase had been pulled off and trapped in a liquid nitrogen cold trap, the vacuum was broken with nitrogen and the heating stopped.

Next, a sample of 1.80 g of the same feed as used initially was added to the stirring clear orange solution remaining in the flask. The mixture was stirred vigorously for 30 minutes, and the two phases were again allowed to separate. A sample of the second raffinate was analyzed by gas chromatography and produced the results shown in Table 7.

TABLE 7

| | | | Percent |
|---|---|---|---|
| Component | Feed | Raffinate | Decrease |
| 2-ethyl-1-hexene | 0.08% | 0.09% | −13.788 |
| 1-octene | 11.98% | 8.66% | 27.733 |
| cis-3-methyl-3-heptene | 0.26% | 0.33% | −27.786 |
| trans-4-octene | 1.21% | 1.18% | 2.425 |
| trans-3-methyl-2-+trans-3- | 3.83% | 3.70% | 3.313 |
| methyl-3-heptene, trans-3-octene | | | |
| trans-2-octene | 4.87% | 4.72% | 3.133 |
| cis-3-methyl-2-heptene | 1.10% | 1.07% | 2.332 |
| cis-2-octene | 3.94% | 3.71% | 5.826 |
| total olefins: | 27.25% | 23.45% | 13.957 |

As can be seen, the composition of the present invention reversibly complexes olefins and removed 28% of the 1-octene and 14% of the total olefins on the second cycle, showing no evidence of deterioration.

Analysis

In summary, the compositions and methods of the present invention are useful for the separation of olefins from various mixtures. Such mixtures may contain olefinic and non-olefinic hydrocarbons. In fact, the methods and compositions of the present invention have been found to be particularly useful for the separation of mixtures of liquid olefins from paraffinic solvents (as are encountered in the production of ethylene-1-octene copolymer). Other streams which are also suitable streams for olefin/paraffin separation are gaseous products from steam cracking and from fluid catalytic cracking.

Without being bound by theory, it is envisioned that the separation processes of the present invention are based on complexation, and more particularly based on the principle that the π electrons in the double bonds of olefins can complex reversibly with transition metal ions, such as Cu⁺. Such reversibility is advantageous because the compositions of the present invention allow the olefins to be de-bonded from the olefins after separation.

By way of background, and without again being bound by theory, a Cu⁺ ion used for a separation may have a coordination number (defined as the number of ligands that can associate with a central metal ion) of 2, 4 or 6, with 4 being the most common. In addition, transition metals like copper have two primary sets of d orbitals that are involved in complex formation. As illustrated in FIG. 2, these are the dₓ²_{-y}² orbitals and the d_{z}² orbitals.

According to crystal field theory, there exists a repulsion between the metal d electrons and electrons in the ligand lone pairs as a ligand approaches the metal atom or ion, causing the d electron orbitals to rise in energy. The largest repulsion is felt by the dₓ²_{-y}² orbitals and the d_{z}² orbitals since they are pointed directly at the incoming ligand electron pairs.

In utilizing the compositions of the present invention, one must consider various attributes of the different components of the present invention. For instance, one attribute is that Ag⁺ is expensive and generally unstable. A second attribute is that Ag⁺ and Cu⁺ transition metals can have significant effects on the behavior of the compositions toward olefins.

A third attribute is that the use of a solvent or ligand with a high vapor pressure (e.g., higher than about 700 torr at the temperature of operation) may affect the olefin separation process. For instance, when such solvents are used in a gas phase absorption process (such as separation of ethylene from ethane or propylene from propane), a portion of that solvent or ligand may become volatilized into the non-absorbed gas stream, thus requiring an additional and costly separation step downstream.

A fourth attribute is that, water, while acceptable as a solvent for Ag⁺ ions, is known to promote the disproportionation of Cu⁺ into Cu⁺⁺ and Cu⁰ if the copper is not adequately coordinated by a ligand. Thus, Cu⁺ may not be suitable for all the metal-ligand combinations of the present invention.

Finally, a fifth attribute is that monodentate nitrogen ligands (like pyridine) are not as effective in stabilizing Cu⁺ as are bidentate or tridentate ligands. Without being bound by theory, it is envisioned that such different stabilities may be based on the principle that the stability of the metal-ligand complexes increase in the following order: monodentate<bidentate<tridentate<tetradentate. Monodentate ligands are generally reversible and tend to have lower boiling points. Therefore, they may not be optimal for use in various embodiments of the present invention. On the other hand, tetradentate ligands stably occupy all coordination sites leaving no room for the olefin. Therefore, the preferred ligands for the compositions of the present invention are bidentate and tridentate ligands.

Finally, one must also keep in mind that, in the absence of a suitable ligand to stabilize it, Cu⁺ will disproportionate into Cu⁺⁺ and Cu⁰, neither of which is capable of binding olefins. Further, a metal ion stabilized by a ligand has been shown to more efficiently complex olefins if it is dissolved in a suitable solvent.

The above attributes and factors were considered in devising the compositions and methods of the present invention for the recovery of olefins from a mixture as claimed in this application. However, based on Applicants' current awareness, such attributes and factors were not considered in the prior art. In addition, Applicants are currently unaware of any similar compositions or methods in the prior art.

From the foregoing description, one skilled in the art can easily ascertain the essential characteristics of this disclosure, and without departing from the spirit and scope thereof, can make various changes and modifications to adapt the disclosure to various usages and conditions.

Therefore, the embodiments described hereinabove are meant to be illustrative only and should not be taken as limiting of the scope of the disclosure, which is defined in the following claims.

What is claimed is:

1. A composition for the recovery of olefins from a mixture, wherein said composition comprises:
a transition metal ion;
a counter anion;
a ligand selected from the group consisting of a bidentate ligand and a tridentate ligand, wherein said ligand comprises at least two nitrogen atoms, and wherein each of said nitrogen atoms comprises a lone pair of electrons; and
a polar solvent with a boiling point of at least about 200° C.

2. The composition of claim 1, wherein said mixture is in a gaseous phase.

3. The composition of claim 1, wherein said mixture is in a liquid phase.

4. The composition of claim 1, wherein said olefin comprises an unsaturated hydrocarbon.

5. The composition of claim 1, wherein said transition metal ion is Cu⁺.

6. The composition of claim 1, wherein said transition metal ion is Ag⁺.

7. The composition of claim 1, wherein said counter anion is selected from the group consisting of PF₆⁻¹, BF₄⁻¹, NO₃⁻¹, BPh₄⁻¹, Cl⁻¹, I⁻¹, Br⁻¹, F⁻¹, and COO⁻.

8. The composition of claim 1, wherein said ligand is a bidentate ligand.

9. The composition of claim 8, wherein said bidentate ligand has a boiling point of at least about 200° C.

10. The composition of claim 8, wherein said bidentate ligand has a vapor pressure of less than about 0.01 kPa at 20° C.

11. The composition of claim 8, wherein said bidentate ligand comprises at least two aromatic rings, and wherein each of said aromatic rings comprises a nitrogen atom with a lone pair of electrons.

12. The composition of claim 8, wherein said bidentate ligand is selected from the group consisting of 2,2′-dipyridyl amine, 2,2′-dipyridyl ketone and 2,2′-dipyridyl methane.

13. The composition of claim 1, wherein said ligand is a tridentate ligand.

14. The composition of claim 13, wherein said tridentate ligand has a boiling point of at least about 200° C.

15. The composition of claim 13, wherein said tridentate ligand has a vapor pressure of less than about 0.01 kPa at 20° C.

16. The composition of claim 13, wherein said tridentate ligand comprises at least two aromatic rings, and wherein each of said aromatic rings comprises a nitrogen atom with a lone pair of electrons.

17. The composition of claim 13, wherein said tridentate ligand is selected from the group consisting of terpyridine and di-(2-picolylamine).

18. The composition of claim 1, wherein said solvent has a vapor pressure of less than about 0.01 kPa at 20° C.

19. The composition of claim 1, wherein said solvent comprises a polyalkylene glycol with a general structure of H—(O—CH₂CH₂)ₙ—OH, wherein n represents a value ranging from 2 to 10.

20. The composition of claim 19, wherein said polyalkylene glycol is selected from the group consisting of diethylene glycol, triethylene glycol, tetraethylene glycol, pentaethylene glycol and hexaethylene glycol.

21. The composition of claim 1, wherein said solvent comprises an ionic liquid.

22. The composition of claim 21, wherein said ionic liquid is selected from the group consisting of 1-butyl-3-methylimidazolium hexafluorophosphate, 1-ethyl-3-methylimidazolium tetrachloroaluminate, 1-butylpyridinium nitrate, 1-butyl-3-methylimidazolium tetrafluoroborate and mixtures thereof.

23. The composition of claim 1, wherein the boiling point of said solvent is higher than the boiling point of the highest boiling olefin in said mixture.

24. The composition of claim 23, wherein the boiling point of said solvent is at least about 20° C. higher than the boiling point of the highest boiling olefin in said mixture.

25. The composition of claim 23, wherein the boiling point of said solvent is at least about 50° C. higher than the boiling point of the highest boiling olefin in said mixture.

26. The composition of claim 23, wherein the boiling point of said solvent is at least about 100° C. higher than the boiling point of the highest boiling olefin in said mixture.

27. A method for recovering olefins from a mixture, wherein said method comprises:
providing a composition that comprises:
a transition metal ion;
a counter anion;
a ligand selected from the group consisting of a bidentate ligand and a tridentate ligand, wherein said ligand comprises at least two nitrogen atoms, and wherein each of said nitrogen atoms comprises a lone pair of electrons; and
a polar solvent with a boiling point of at least about 200° C.;
bonding at least a portion of said olefins in said mixture to said transition metal ion in said composition to form a complex;
separating said complex from said mixture; and
recovering said olefins from said complex.

28. The method of claim 27, wherein said transition metal ion in said composition is Cu⁺.

29. The method of claim 27, wherein said ligand in said composition is a bidentate ligand with at least two aromatic rings, wherein each of said aromatic rings comprises a nitrogen atom with a lone pair of electrons.

30. The method of claim 27, wherein said ligand in said composition is a tridentate ligand with at least two aromatic rings, wherein each of said aromatic rings comprises a nitrogen atom with a lone pair of electrons.

31. The method of claim 27, wherein said bonding comprises mixing said composition with said mixture.

32. The method of claim 31, wherein said mixing comprises stiffing.

33. The method of claim 27, wherein said separation comprises phase separation.

34. The method of claim 33, wherein said phase separation comprises incubating said complex and said mixture at room temperature.

35. The method of claim 33, wherein said phase separation comprises centrifugation.

36. The method of claim 27, wherein said recovery comprises reducing pressure.
